(19) United States
(12) Patent Application Publication
(10) Pub. No.: US 2026/0137761 A1
(43) Pub. Date: May 21, 2026

(54) INJECTION COMPOSITION FOR LIPOLYSIS

(71) Applicant: Min Jae KANG, Daegu (KR)

(72) Inventor: Min Jae KANG, Daegu (KR)

(21) Appl. No.: 19/339,883

(22) Filed: Sep. 25, 2025

(30) Foreign Application Priority Data
Nov. 21, 2024 (KR) 10-2024-0167024

(51) Int. Cl. A61K 38/47; A61K 9/00; A61K 31/167; A61K 31/197; A61K 31/522; A61P 3/04

(52) U.S. Cl. CPC A61K 38/47; A61K 9/0019; A61K 31/167; A61K 31/197; A61K 31/522; A61P 3/04

(57) ABSTRACT

The present disclosure relates to an injection composition for lipolysis with both safety and efficacy, and more particularly, to an injection composition for lipolysis including at least one compound hyaluronidase and pentoxifylline; 5 to 10% (v/v) carnitine; lidocaine; and a physiological saline. The injection composition for lipolysis according to the present disclosure is a composition with both safety and efficacy, which is characterized by having an excellent lipolysis effect, and having not only no yo-yo effect over time, but also no side effects such as sagging skin.

### CROSS-REFERENCE TO RELATED APPLICATION

This application claims the priority of Korean Patent Application No. 10-2024-0167024 filed on Nov. 21, 2024, in the Korean Intellectual Property Office, the disclosure of which is incorporated herein by reference.

### BACKGROUND

### Field

The present disclosure relates to an injection composition for lipolysis with both safety and efficacy, and more particularly, to an injection composition for lipolysis including at least one compound of hyaluronidase and pentoxifylline; 5 to 10% (v/v) carnitine; lidocaine; and a physiological saline.

### Description of the Related Art

According to a World Health Organization (WHO) report, obesity rates have been rapidly increasing since 1975 around high-income countries and are also showing a similar tendency in Korea. As the Korean economy has advanced, living standards have improved and dietary life has changed, but due to a decrease in physical activity and an increase in high-calorie food intake, obesity rates have also been steadily on the rise. An increase in the obese population causes economic, social, and cultural burdens beyond simple physical problems, and consequently, the costs of the treatment for various obesity-related diseases are also increasing significantly. The economic burden associated with obesity treatment is currently estimated to reach tens of billions of dollars annually, and experts predict that the costs will continue to rise (Global Pipeline Analysis, Competitive Landscape, and Market Forecasts).

In the past, liposuction and various lipolysis procedures were primarily used for the treatment of obesity. However, these existing procedures not only caused severe pain to patients, but also resulted in various side effects, such as adhesion to the dermal layer, hematoma formation, and infection at the treatment area. In particular, in some early procedures, there were reported cases in which potentially carcinogenic substances were used, which ran a risk of serious health problems. To solve these problems, the medical community is continuously researching new treatments capable of maximizing lipolysis effect while minimizing pain and side effects. This research is developing to shorten a post-procedure recovery period and reduce the risk of local tissue necrosis and infection through minimally invasive approaches.

As awareness of body types becomes increasingly important in modern society, aesthetic demands for body type improvement and body contouring are increasing beyond simple obesity treatment. Accordingly, the development of an injection composition for lipolysis, which is more effective and safer and has minimal side effects, has emerged as a critical issue. Particularly, as the number of consumers requiring body type improvement through lipolysis increases, these compositions need to meet consumer demands with both safety and efficacy, and current ongoing studies have been developed to reflect these market demands.

Therefore, modern compositions for lipolysis need to be developed as a safe method for not only meeting economic and social needs but also enhancing user satisfaction. Ultimately, these compositions are anticipated to be effective and reliable alternatives for obesity treatment and body type management and are expected to continue to be developed in the future.

### PRIOR ARTS

### Patent Documents

(Patent Document 1) Korean Patent Registration No. 10-2517928
(Patent Document 2) Korean Patent Registration No. 10-2627595

### SUMMARY

An object of the present disclosure is to provide an injection composition for lipolysis including: at least one compound of hyaluronidase and pentoxifylline; 5 to 10% (v/v) carnitine; 0.1 to 1.0% (v/v) lidocaine; and a physiological saline.

In order to achieve the object, the present disclosure provides an injection composition for lipolysis including: at least one compound of hyaluronidase and pentoxifylline; 5 to 10% (v/v) carnitine; 0.1 to 1.0% (v/v) lidocaine; and a physiological saline.

When the injection composition for lipolysis includes hyaluronidase, the hyaluronidase content may be included in units of 600 IU to 900 IU.

When the injection composition for lipolysis includes pentoxifylline, the pentoxifylline content may be included in 8 to 12% (v/v).

The injection composition may further include one or more additives selected from the group consisting of excipients, permeation enhancers, dispersants, disintegrants, lubricants, wetting agents, emulsifiers, suspending agents, diluents, binders, slip modifiers, antiseptics, stabilizers, anti-adhesives, fluidizing agents, colorants, or preservatives.

The injection composition may have a single injection dose of 1.0 to 15.0 cc.

The injection composition may be administered at injection point intervals of 1.5 to 2.5 cm.

The injection composition for lipolysis may have no side effects of sagging skin.

According to the present disclosure, the injection composition for lipolysis is a composition with both safety and efficacy, which is characterized by having an excellent lipolysis effect and having not only no yo-yo effect over time, but also no side effects such as sagging skin.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### DETAILED DESCRIPTION

Hereinafter, the present disclosure will be described in more detail through embodiments. These embodiments are to explain the present disclosure in more detail, and it will be apparent to those skilled in the art that the scope of the present disclosure is not limited by these embodiments in accordance with the gist of the present disclosure.

The present disclosure provides an injection composition for lipolysis including: at least one compound of hyaluronidase and pentoxifylline; 5 to 10% (v/v) carnitine; 0.1 to 1.0% (v/v) lidocaine; and a physiological saline.

In the present disclosure, the injection composition for lipolysis may include hyaluronidase.

The hyaluronidase is an enzyme that breaks down hyaluronic acid, and corresponds to an ingredient that provides functions, such as reduced edema, lipolysis, accelerated drug absorption when injected, etc.

In the present disclosure, when the injection composition for lipolysis includes hyaluronidase, the hyaluronidase content may be included in units of 600 IU to 900 IU. At this time, when the hyaluronidase is included in less than 600 IU, a lipolysis effect may not be sufficient and a yo-yo effect may occur, resulting in a decrease in a waist circumference reduction effect, and when the hyaluronidase is included in more than 900 IU, not only the yo-yo effect may occur, but also side effects such as sagging skin may occur. Accordingly, when the injection composition for lipolysis includes hyaluronidase, the injection composition exhibits both excellent lipolysis effect and safety when the hyaluronidase is included in an amount of 600 IU to 900 IU.

In the present disclosure, the injection composition for lipolysis may include pentoxifylline.

The pentoxifylline is an ingredient that lowers blood viscosity and promotes blood circulation, thereby helping to supply more oxygen to tissues, and provides a lipolysis effect by activating adipocyte metabolism and improving blood circulation.

In the present disclosure, when the injection composition for lipolysis includes pentoxifylline, the pentoxifylline may be included in 8 to 12% (v/v) based on the total volume of the injection composition. At this time, when the pentoxifylline is included in less than 8% (v/v), there may be problems that the waist circumference reduction effect may be reduced and the yo-yo effect may occur significantly, and when the pentoxifylline is included in more than 12% (v/v), there may be problems that side effects such as urticaria, dizziness, and nausea may occur. On the other hand, when the pentoxifylline is included in 8 to 12% (v/v), the waist circumference reduction effect may be improved and the yo-yo effect may be reduced.

In the present disclosure, the injection composition for lipolysis may include carnitine.

The carnitine may be selected from the group consisting of L-carnitine, acetyl-L-carnitine (ALCAR), propionyl-L-carnitine, D-carnitine, L-carnitine L-tartrate, glycine propionyl-L-carnitine (GPLC), and combinations thereof, but is not limited thereto.

Carnitine is an amino acid derivative required for the body to convert fat into energy, and corresponds to an ingredient that aids in fat burning, energy production, body fat reduction, improved exercise performance, etc.

In the present disclosure, the injection composition for lipolysis may include the carnitine in an amount of 5 to 10% (v/v) based on the total volume of the injection composition. At this time, when the carnitine is included in less than 5% (v/v), the lipolysis effect may not be sufficiently exhibited, and when the carnitine is included in more than 10% (v/v), side effects such as sagging skin and excessive yo-yo effect may occur.

In the present disclosure, the injection composition for lipolysis includes lidocaine.

Lidocaine is a local anesthetic ingredient and used to alleviate pain when injecting the injection composition.

In the present disclosure, the injection composition for lipolysis may include the lidocaine in an amount of 0.1 to 1.0% (v/v) based on the total volume of the injection composition. At this time, when the lidocaine is included in less than 0.1% (v/v), the injection composition may cause pain or discomfort, and the ingredient of the injection composition may not be effective at an injection area, and when the lidocaine is included in more than 1%, various side effects such as cardiovascular side effects and skin irritation may occur.

In the present disclosure, the composition of the present disclosure may further include suitable additives, such as carriers, excipients, permeation enhancers, dispersants, disintegrants, lubricants, wetting agents, emulsifiers, suspending agents, diluents, binders, slip modifiers, antiseptics, stabilizers, anti-adhesives, fluidizing agents, colorants, or preservatives, which are commonly used in the preparation of the injection composition. The carriers, the excipients, and the diluents that may be included in the composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, saline solution, methyl hydroxybenzoate, propyl hydroxybenzoate, talc, magnesium stearate, mineral oils, etc. When the composition is formulated, the formulations may be prepared by using diluents or excipients, such as a filler, an extender, a binder, a wetting agent, a disintegrating agent, and a surfactant, which are generally used.

In the present disclosure, the term ‘administration’ used herein means providing a predetermined composition of the present disclosure to a subject or patient through injection. The administration method may include subcutaneous injection, intradermal injection, intravenous injection, intramuscular injection, intraperitoneal injection, subcutaneous implantation, implantable injection, ointments, or patches, and preferably, may be a method for injecting a drug into the dermal and subcutaneous layers.

In addition, the injection composition for lipolysis may be injected using an infusion pump. In particular, when an excessive amount of the injection composition for lipolysis is administered, the injection composition may be appropriately injected using an infusion pump other than a syringe.

In the present disclosure, the injection composition for lipolysis may be administered to an area where the lipolysis effect is desired, such as the forearm, abdomen, thigh, face, or buttocks.

In the present disclosure, a preferable dose of the injection composition for lipolysis varies according to a subject, the condition and body weight of a patient, the presence or absence of disease, the severity of a disease, a route of administration, and duration of administration, but may be properly selected by those skilled in the art. For a desirable effect, the injection composition of the present disclosure may be administered in an amount of 1.0 to 15.0 cc once, and may also be administered once or several divided times a day.

In the present disclosure, injection points of the injection composition for lipolysis may be intervals of 1.5 to 2.5 cm, but are not limited thereto.

In the present disclosure, the injection composition for lipolysis not only exhibits an excellent lipolysis effect, but also exhibits a slight yo-yo effect even after one month of administration of the composition and has no side effects such as sagging skin.

Hereinafter, the present disclosure will be described in detail by Examples. However, the following Examples are just illustrative of the present disclosure, and the contents of the present disclosure are not limited to the following Examples.

### Example 1

Hyaluronidase (purchased from Daehan New Pharm), carnitine (purchased from Dongkwang Pharmaceutical), and lidocaine (purchased from Daehan New Pharm) were uniformly mixed in the contents shown in Table 1 below and dissolved in a physiological saline to prepare an injection composition for lipolysis of Example 1.

TABLE 1

| Ingredient | Example 1 |
|---|---|
| Hyaluronidase | 700 IU |
| L-carnitine | 10% (v/v) |
| Lidocaine | 0.6% (v/v) |
| Physiological saline | Residue |

### Example 2

Pentoxifylline (purchased from Huons), carnitine (purchased from Dongkwang Pharmaceutical), and lidocaine (purchased from Daehan New Pharm) were uniformly mixed in the contents shown in Table 2 below and dissolved in a physiological saline to prepare an injection composition for lipolysis of Example 2.

TABLE 2

| Ingredient | Example 2 |
|---|---|
| Pentoxifylline | 10% (v/v) |
| L-carnitine | 10% (v/v) |
| Lidocaine | 0.6% (v/v) |
| Physiological saline | Residue |

### Example 3

Hyaluronidase (purchased from Daehan New Pharm), pentoxifylline (purchased from Huons), carnitine (purchased from Dongkwang Pharmaceutical), and lidocaine (purchased from Daehan New Pharm) were uniformly mixed in the contents shown in Table 3 below and dissolved in a physiological saline to prepare an injection composition for lipolysis of Example 3.

TABLE 3

| Ingredient | Example 2 |
|---|---|
| Hyaluronidase | 700 IU |
| Pentoxifylline | 10% (v/v) |
| L-carnitine | 10% (v/v) |
| Lidocaine | 0.6% (v/v) |
| Physiological saline | Residue |

### Comparative Example 1

Compared to the injection composition for lipolysis of Example 1, there was only a difference that 500 IU of hyaluronidase was included instead of 700 IU of hyaluronidase, but other ingredients and contents were included in the same manner as Example 1 and then an injection composition for lipolysis of Comparative Example 1 was prepared.

### Comparative Example 2

Compared to the injection composition for lipolysis of Example 1, there was only a difference that 1000 IU of hyaluronidase was included instead of 700 IU of hyaluronidase, but other ingredients and contents were included in the same manner as Example 1 and then an injection composition for lipolysis of Comparative Example 2 was prepared.

### Comparative Example 3

Compared to the injection composition for lipolysis of Example 2, there was only a difference that 5% (v/v) pentoxifylline was included instead of 10% (v/v) pentoxifylline, but other ingredients and contents were included in the same manner as Example 2 and then an injection composition for lipolysis of Comparative Example 3 was prepared.

### Comparative Example 4

Compared to the injection composition for lipolysis of Example 2, there was only a difference that 15% (v/v) pentoxifylline was included instead of 10% (v/v) pentoxifylline, but other ingredients and contents were included in the same manner as Example 2 and then an injection composition for lipolysis of Comparative Example 4 was prepared.

### Comparative Example 5

Compared to the injection composition for lipolysis of Example 3, there was only a difference that 5% (v/v) pentoxifylline was included instead of 10% (v/v) pentoxifylline, but other ingredients and contents were included in the same manner as Example 3 and then an injection composition for lipolysis of Comparative Example 5 was prepared.

### Comparative Example 6

Compared to the injection composition for lipolysis of Example 3, there was only a difference that 1,000 IU of hyaluronidase was included instead of 700 IU of hyaluronidase, and 5% (v/v) pentoxifylline was included instead of 10% (v/v) pentoxifylline, but other ingredients and contents were included in the same manner as Example 3 and then an injection composition for lipolysis of Comparative Example 6 was prepared.

### Comparative Example 7

Compared to the injection composition for lipolysis of Example 3, there was only a difference that 15% (v/v) pentoxifylline was included instead of 10% (v/v) pentoxifylline, but other ingredients and contents were included in the same manner as Example 3 and then an injection composition for lipolysis of Comparative Example 7 was prepared.

The ingredients and the contents of the injection compositions of Examples 1 to 3 and Comparative Examples 1 to 7 were summarized and shown in Table 4 below.

TABLE 4

| | | | | | Physiological |
|---|---|---|---|---|---|
| Classification | Hyaluronidase | Pentoxifylline | L-carnitine | Lidocaine | saline |
| Example 1 | 700 IU | — | 10% (v/v) | 0.6% (v/v) | Residue |
| Example 2 | — | 10% (v/v) | 10% (v/v) | 0.6% (v/v) | |
| Example 3 | 700 IU | 10% (v/v) | 10% (v/v) | 0.6% (v/v) | |
| Comparative | 500 IU | — | 10% (v/v) | 0.6% (v/v) | |
| Example 1 | | | | | |
| Comparative | 1000 IU | — | 10% (v/v) | 0.6% (v/v) | |
| Example 2 | | | | | |
| Comparative | — | 5% (v/v) | 10% (v/v) | 0.6% (v/v) | |
| Example 3 | | | | | |
| Comparative | — | 15% (v/v) | 10% (v/v) | 0.6% (v/v) | |
| Example 4 | | | | | |
| Comparative | 700 IU | 5% (v/v) | 10% (v/v) | 0.6% (v/v) | |
| Example 5 | | | | | |
| Comparative | 1000 IU | 5% (v/v) | 10% (v/v) | 0.6% (v/v) | |
| Example 6 | | | | | |
| Comparative | 700 IU | 15% (v/v) | 10% (v/v) | 0.6% (v/v) | |
| Example 7 | | | | | |

### Experimental Example 1. Clinical Trials

To confirm the lipolysis effects of injection compositions for lipolysis of Examples 1 to 3 and Comparative Examples 1 to 7, clinical trials were conducted on 10 male and female patients aged 20 years or older with abdominal obesity having BMI 25 or higher. 10 cc of the injection composition for lipolysis was administered into the abdomen of each subject at 2 cm intervals, and then a thickness (mm) of the injected abdominal fat administered with the injection composition was measured on day 3 and month 1 after administration, respectively, and the results were shown in Table 5 below. At this time, a difference in waist circumference after 1 month of administration meant a change occurring based on the waist circumference measured on day 3 after administration.

Meanwhile, a distribution of measurements was obtained using an SPSS statistical package by statistical analysis and verification methods, and a paired t-test was used to determine an abdominal waist circumference reduction effect of each experimental injection, and a reliability verification interval was set within 95%.

TABLE 5

| | Difference in waist | Difference in waist | |
|---|---|---|---|
| | circumference after 3 | circumference after | Occurrence of side |
| | days of | 1 month of | effects such as |
| | administration | administration | sagging skin, |
| | (mm) | (mm) | dizziness, etc. |
| Example 1 | −4.3 ± 0.3 | +3.3 ± 0.5 | Absence |
| Example 2 | −3.2 ± 0.2 | −0.8 ± 0.2 | Absence |
| Example 3 | −4.7 ± 0.2 | −1.3 ± 0.1 | Absence |
| Comparative | −1.9 ± 0.3 | +1.6 ± 0.1 | Absence |
| Example 1 | | | |
| Comparative | −4.8 ± 0.5 | +4.5 ± 0.5 | Sagging skin occurred |
| Example 2 | | | |
| Comparative | −2.4 ± 0.8 | −0.4 ± 0.6 | Absence |
| Example 3 | | | |
| Comparative | −3.7 ± 0.6 | −0.9 ± 0.3 | Urticaria, dizziness, |
| Example 4 | | | and nausea occurred |
| Comparative | −3.8 ± 0.1 | −0.2 ± 0.2 | Absence |
| Example 5 | | | |
| Comparative | −5.2 ± 0.3 | +4.4 ± 0.2 | Sagging skin occurred |
| Example 6 | | | |
| Comparative | −4.5 ± 1.7 | −1.0 ± 0.4 | Urticaria, dizziness, |
| Example 7 | | | and nausea occurred |

As the results of clinical trials confirming the lipolysis effect, it was confirmed that the injection compositions for lipolysis according to Examples 1 to 3 of the present disclosure not only exhibited excellent waist circumference reduction effects, but also caused no side effects such as sagging skin, urticaria, and dizziness. In particular, the injection compositions for lipolysis of Examples 2 and 3 did not exhibit a yo-yo effect even up to 1 month after administration, but exhibited a sustained lipolysis effect.

On the other hand, the injection composition for lipolysis of Comparative Example 1 containing 500 IU of hyaluronidase not only exhibited no significant waist circumference reduction effect 3 days after administration, but also exhibited a yo-yo effect after 1 month of administration to have almost no lipolysis effect. The injection composition for lipolysis of Comparative Example 2 containing 1,000 IU of hyaluronidase not only exhibited a yo-yo effect, but also caused finely sagging skin due to an excessive dose of hyaluronidase. That is, when confirming a difference in waist circumference according to the hyaluronidase content, it was confirmed that the lipolysis effect was not sufficiently exhibited when the hyaluronidase content was 500 IU or less, and it was confirmed that finely sagging skin occurred to potentially cause minor side effects when the hyaluronidase content was 1,000 IU or more.

In addition, the injection composition for lipolysis of Example 2 containing 10% (v/v) pentoxifylline as a lipolysis compound not only exhibited a superior lipolysis effect, but also maintained the lipolysis effect even after 1 month and exhibited superior durability, compared to the injection composition of Comparative Example 3 containing 5% (v/v) pentoxifylline. In addition, it was confirmed that the injection composition for lipolysis of Comparative Example 4 containing 15% (v/v) pentoxifylline caused side effects such as nausea, dizziness, and urticaria, compared to the injection composition for lipolysis of Example 2 containing 10% (v/v) pentoxifylline as a lipolysis compound. That is, when the content of pentoxifylline is 5% or less, the lipolysis effect may have a yo-yo effect and a decrease in the lipolysis effect may occur, and when the content of pentoxifylline is 15% or more, side effects such as urticaria, dizziness, and nausea may occur.

Meanwhile, in Comparative Examples 5 to 7, containing both hyaluronidase and pentoxifylline as lipolysis compounds, Comparative Example 5 exhibited no yo-yo effect, but the content of pentoxifylline was insufficient, so that the waist circumference reduction effect was very slight after 1 month of administration. In other words, the injection composition of Comparative Example 5 was found to have a poor persistence of the lipolysis effect. The injection composition of Comparative Example 6 showed a significant decrease in waist circumference 3 days after administration, but it was confirmed that the waist circumference after 1 month was almost the same as the waist circumference before the procedure, and thus the yo-yo effect was severe, which was confirmed to be a temporary edema or swelling reduction effect according to an excessive hyaluronidase content. In addition, the injection composition of Comparative Example 7 contained an excessive content of pentoxifylline, so that side effects such as urticaria, dizziness, and nausea were observed. That is, in the case of containing both hyaluronidase and pentoxifylline as the lipolysis compounds, when the content of hyaluronidase was included in 700 IU and pentoxifylline was included in 10% (v/v), it was confirmed that the injection composition for lipolysis had an excellent lipolysis effect and safety.

What is claimed is:

1. An injection composition for lipolysis comprising: at least one compound of hyaluronidase and pentoxifylline; 5 to 10% (v/v) carnitine; 0.1 to 1.0% (v/v) lidocaine; and a physiological saline.

2. The injection composition for lipolysis of claim 1, wherein when the injection composition for lipolysis includes the hyaluronidase, a content of the hyaluronidase is included in units of 600 IU to 900 IU.

3. The injection composition for lipolysis of claim 1, wherein when the injection composition for lipolysis includes the pentoxifylline, a content of the pentoxifylline is included in 8 to 12% (v/v).

4. The injection composition for lipolysis of claim 1, wherein the injection composition further includes one or more additives selected from the group consisting of excipients, permeation enhancers, dispersants, disintegrants, lubricants, wetting agents, emulsifiers, suspending agents, diluents, binders, slip modifiers, antiseptics, stabilizers, anti-adhesives, fluidizing agents, colorants, or preservatives.

5. The injection composition for lipolysis of claim 1, wherein the injection composition has a single injection dose of 15 cc.

6. The injection composition for lipolysis of claim 1, wherein the injection composition is administered at injection point intervals of 1.5 to 2.5 cm.

7. The injection composition for lipolysis of claim 1, wherein the injection composition for lipolysis has no side effects of sagging skin.

8. A method for inducing lipolysis in a subject in need thereof, the method comprising administering to a dermal or subcutaneous layer of the subject the injection composition for lipolysis according to claim 1.
